(19) United States
(12) Patent Application Publication
(10) Pub. No.: US 2024/0166979 A1
(43) Pub. Date: May 23, 2024

(54) CULTURE DEVICE

(71) Applicant: HONDA MOTOR CO., LTD., MINATO-KU, TOKYO (JP)

(72) Inventors: Minoru GOTO, WAKO-SHI, SAITAMA-KEN (JP); Nozomi SHIOBARA, WAKO-SHI, SAITAMA-KEN (JP); Shohei KINOSHITA, WAKO-SHI, SAITAMA-KEN (JP); Fumitomo TAKANO, WAKO-SHI, SAITAMA-KEN (JP); Mizuho DOI, WAKO-SHI, SAITAMA-KEN (JP); Satoshi SHIOZAKI, WAKO-SHI, SAITAMA-KEN (JP); Kenji MACHIDA, WAKO-SHI, SAITAMA-KEN (JP)

(21) Appl. No.: 18/281,707

(22) PCT Filed: Feb. 24, 2022
(86) PCT No.: PCT/JP2022/007451

§ 371 Date: Sep. 12, 2023

(30) Foreign Application Priority Data
Mar. 15, 2021 (JP) 2021-041556

(51) Int. Cl. C12M 1/00; C12M 1/02

(52) U.S. Cl. CPC C12M 21/02; C12M 23/22; C12M 41/20

(57) ABSTRACT

A culture device is provided with a culture tank having sidewalls that are formed from a translucent material, and cultures fine algae in a culture liquid that is accommodated in the culture tank and through which light is emitted via the sidewalls. The culture device is provided with a translucent insulating part that covers the sidewalls. The insulating part forms an air layer that insulates the inside of the culture tank. A culture device is provided with a culture tank having sidewalls that are formed from a translucent material, and cultures fine algae in a culture liquid that is accommodated in the culture tank and through which light is emitted via the sidewalls. The culture device is provided with a translucent insulating part that covers the sidewalls. The insulating part forms an air layer that insulates the inside of the culture tank.

### TECHNICAL FIELD

The present invention relates to a culturing device (culture device) for culturing microalgae.

### BACKGROUND ART

For example, a culturing device, as disclosed in JP 2014-516550 A, is known. Such a culturing device stores a culturing solution in a culturing tank which is made up from a V-shaped trough having an upper opening. In such a culturing device, microalgae are cultured in the culturing solution while light is shone in the depth direction of the culturing solution primarily from the upper opening of the culturing tank. In such a culturing device, a foam heat insulating material is adhered to the side walls of the culturing tank in order to increase the heat insulation property inside the culturing tank. In accordance with this feature, the culturing solution inside the culturing tank can be prevented from receiving an influence of changes in the external environment (for example, the outside air temperature or the intensity of solar radiation). As a result, it is easy to maintain the culturing solution at a temperature suitable for culturing the microalgae. Consequently, it becomes possible for the microalgae to be satisfactorily cultured.

### SUMMARY OF THE INVENTION

Incidentally, a culturing device has been proposed that includes a culturing tank having side walls that have the property of transmitting light. In such a culturing device, the microalgae are cultured while the microalgae are irradiated through the side walls with light from a direction that intersects a depth direction of the culturing solution. In this type of culturing device, for example, in comparison with a culturing device that shines light in the depth direction of the culturing solution through an upper opening, it is possible to easily increase the ratio of the light receiving area with respect to the volume in which the microalgae are cultured. As a result, it is possible to distribute the light energy, in which an excess or deficiency of light is suppressed, to a greater amount of the microalgae inside a culturing tank. Therefore, it becomes possible for the microalgae to be satisfactorily cultured.

In the culturing device in which light is shone into the culturing tank through the side walls, it may be considered to adhere a foam heat insulating material to the side walls in order to enhance the heat insulation property inside the culturing tank. However, when the foam insulation is adhered to the side walls, the foam insulation blocks the light shone through the side walls. For this reason, it has been difficult to increase the heat insulation property inside the culturing tank, while at the same time increasing the ratio of the light receiving area with respect to the volume in which the microalgae are cultured.

The present invention has the object of solving the aforementioned problem.

One aspect of the present invention is a culturing device including a culturing tank the side walls of which are made from a light-transmitting material, and configured to culture microalgae in a culturing solution accommodated in the culturing tank and which is irradiated with light through the side walls, and also including a light-transmitting heat insulating portion configured to cover the side walls, wherein the heat insulating portion forms an air layer that insulates the interior of the culturing tank.

In such a culturing device, the side walls of the light-transmitting culturing tank are covered by a light-transmitting heat insulating portion. Therefore, the interior of the culturing tank is insulated by an air layer which is formed by the heat insulating portion. In this case, the interior of the culturing tank can be satisfactorily irradiated with light through the heat insulating portion, the air layer, and the side walls, all of which have the light-transmitting property. As a result, it is possible to increase the heat insulation property inside the culturing tank, while at the same time increasing the ratio of the light receiving area with respect to the volume in which the microalgae are cultured. Therefore, it becomes possible for the microalgae to be satisfactorily cultured.

### BRIEF DESCRIPTION OF THE DRAWINGS

FIG. 1 is a schematic front view of a culturing device according to an embodiment of the present invention;

FIG. 2 is a schematic front view for explaining a heat insulating portion and a support mechanism of the culturing device shown in FIG. 1;

FIG. 3 is a schematic side view of the culturing device for describing a state in which an air layer is made thinner by the support mechanism shown in FIG. 2;

FIG. 4 is a schematic side view of the culturing device for describing a state in which the air layer is made thicker by the support mechanism shown in FIG. 2;

FIG. 5 is a schematic front view of a culturing tank of the culturing device shown in FIG. 1;

FIG. 6 is a cross-sectional view taken along line VI-VI of FIG. 5;

FIG. 7 is a schematic perspective view of a liquid storage tank of the culturing device shown in FIG. 1; and

FIG. 8 is a schematic side view of the culturing device for describing a heat insulating portion according to an exemplary modification.

### DESCRIPTION OF THE INVENTION

In the drawings described below, constituent elements having the same or similar functions and effects are designated by the same reference numerals, and repeated description of such features may be omitted.

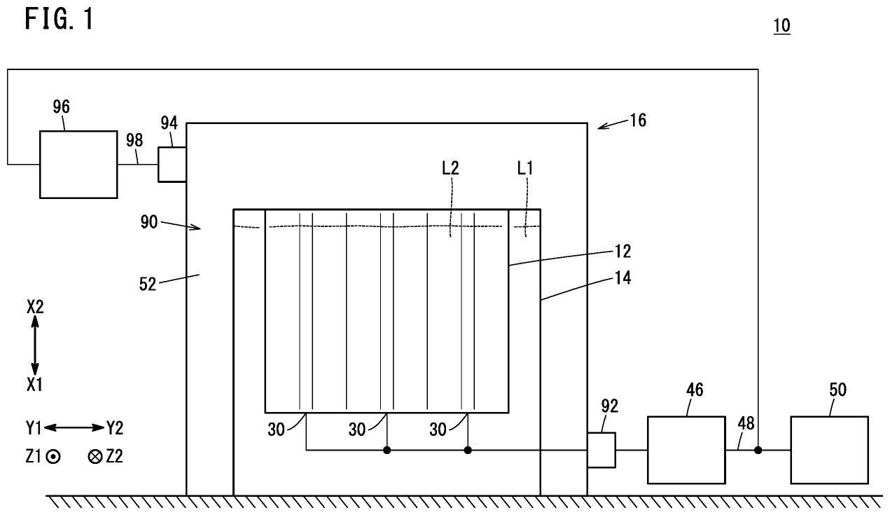

A culturing device 10 according to the present embodiment shown in FIG. 1 supplies light and a supplied gas to microalgae existing within a culturing solution L2 that contains water. As the supplied gas, there may be cited, for example, carbon dioxide gas or a carbon dioxide containing gas (for example, air). Consequently, in the culturing device 10, the microalgae propagate while carrying out photosynthesis. Stated otherwise, the culturing device 10 cultures the microalgae. Moreover, in addition to water, the culturing solution L2 contains necessary nutrients (for example, nitrogen, phosphorus, and potassium) for culturing the microalgae. The supplied gas preferably contains a carbon dioxide gas discharged from a factory or the like.

The microalgae that are capable of being cultured by the culturing device 10 are not particularly limited. In the case of using the cultured microalgae to produce, for example, a biofuel such as ethanol, it is preferable to carry out culturing by the culturing device 10 of microalgae which are classified into chlorophyceae (chlamydomonas and chlorella), prasinophyceae, cryptophyceae, and cyanophyceae (e.g., spirulina). As a suitable example of the microalgae cultured by the culturing device 10, there may be cited the “Honda DREAMO strain” deposited at the National Institute for Product Evaluation Technology Patent Microorganism Depositary Center (Kisarazu City, Chiba Prefecture, Japan, Kazusa Kamatari 2-5-8 Room 120) (the date of deposit thereof is Apr. 22, 2016, and the accession number is FERM BP-22306).

The culturing device 10 is installed in an environment in which the microalgae can be irradiated with light having a wavelength (for example, 400 to 700 nm) required for growth of the microalgae. As an example of such an environment, there may be cited an outdoor environment where sunlight can be irradiated onto the microalgae. However, the culturing device 10 may be installed, for example, inside a room where the microalgae can be irradiated with sunlight or artificial light.

Hereinafter, regarding the orientation of each of the respective constituent elements of the culturing device 10, as shown in FIG. 1, for a case in which the culturing device 10 is installed at an installation location where culturing of the microalgae is to be carried out, descriptions will be made with reference to a vertical direction (the direction of the arrows X1 and X2 in FIG. 1), a first horizontal direction (the direction of the arrows Y1 and Y2 in FIG. 1), and a second horizontal direction (the direction of the arrows Z1 and Z2 in FIG. 1) which is perpendicular to the first horizontal direction. Moreover, although not particularly limited thereto, it is preferable that the first horizontal direction is an east-west direction, and the second horizontal direction is a north-south direction.

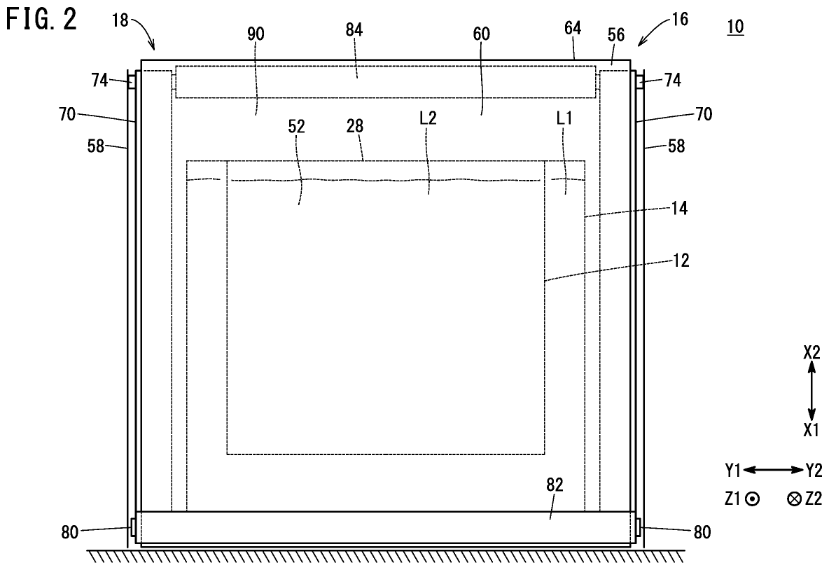

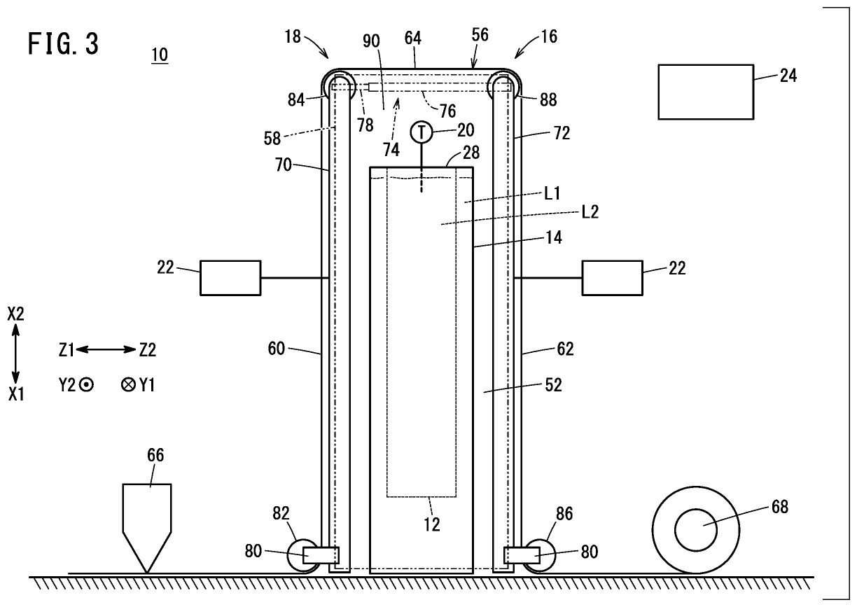

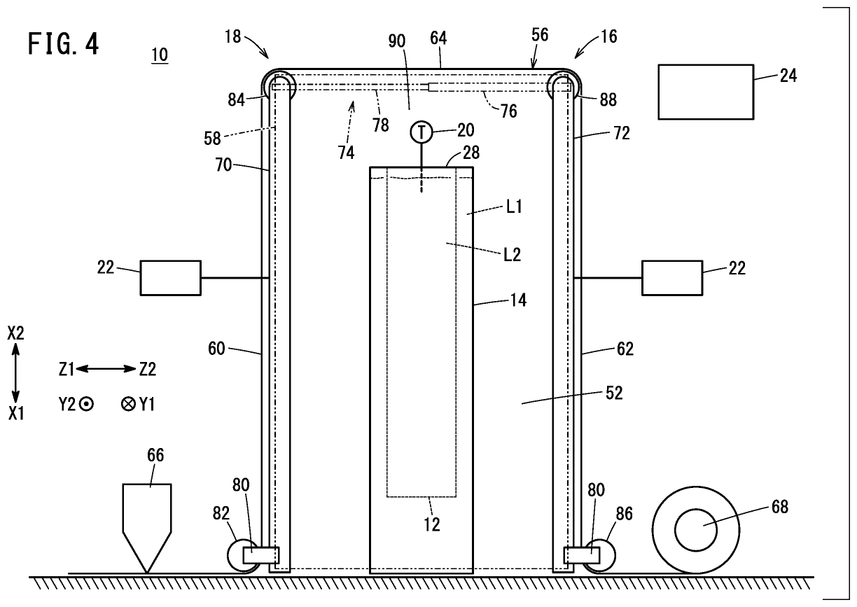

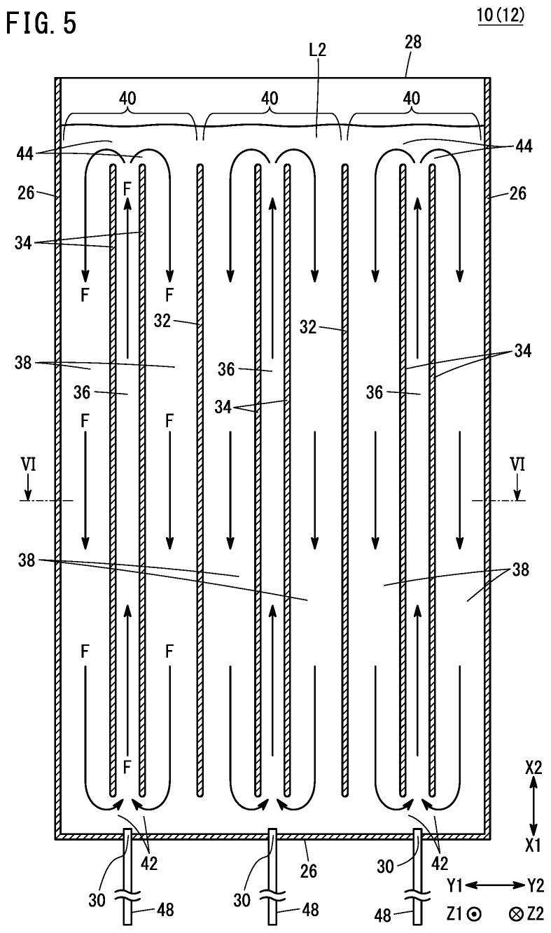

As shown in FIGS. 1 to 4, the culturing device 10 includes a culturing tank 12, a liquid storage tank 14, a heat insulating portion 16, a support mechanism 18 (see FIGS. 2 to 4), a temperature sensor 20 (see FIG. 3 and FIG. 4), a drive unit 22 (see FIG. 3 and FIG. 4), and a control unit 24 (see FIG. 3 and FIG. 4). As shown in FIG. 5, the culturing tank 12 is capable of accommodating the microalgae and the culturing solution L2. The culturing tank 12, for example, is formed from a flexible and light-transmitting material such as linear low density polyethylene (LLDPE). Moreover, in this instance, the term light-transmitting implies that light having a wavelength required for growth of the microalgae can be transmitted through such a material. According to the present embodiment, the culturing tank 12 as a whole is formed from such a light-transmitting material. However, concerning the culturing tank 12, it is sufficient that at least the side surfaces (surfaces excluding the bottom surface and the upper surface) thereof are made of a light-transmitting material

Further, according to the present embodiment, joined edge portions 26 are provided on an outer peripheral edge portion (side portions and a bottom portion) of the culturing tank 12 excluding the upper end. The joined edge portions 26 are formed, for example, by joining inner wall surfaces of the culturing tank 12 to each other by fusion bonding. At the upper end of the culturing tank 12 where the joined edge portions 26 are not provided, an opening is provided that makes it possible to access the interior of the culturing tank 12. Moreover, in FIG. 5, for facilitating the description thereof, the joined locations formed by fusion bonding are shown by hatched lines.

The opening of the culturing tank 12 may always be kept open toward the exterior of the culturing tank 12. The opening of the culturing tank 12 may also be configured so as to be capable of being opened and closed by a non-illustrated opening/closing member. In the case that culturing of the microalgae is carried out with the opening being kept in an open state, the opening serves as a communication port 28 that places the interior and the exterior of the culturing tank 12 in communication with each other. It becomes possible for an exhaust gas to be discharged from the interior to the exterior of the culturing tank 12 via the communication port 28. Further, it becomes possible for the exhaust gas to enter again from the exterior to the interior of the culturing tank 12 via the communication port 28. As an example of the exhaust gas, as will be described later, among the gas supplied to the interior of the culturing tank 12 from the gas supply ports 30, there can be cited a residual gas that is not consumed by photosynthesis of the microalgae. As the exhaust gas, there can also be cited an oxygen containing gas that is generated by photosynthesis.

On the other hand, in the case that the opening of the culturing tank 12 is capable of being opened and closed, for example, the opening may be normally closed. The opening may be open only at a time of accessing the interior of the culturing tank 12, such as at a time when recovering the microalgae from the interior of the culturing tank 12. In this manner, in the case that culturing of the microalgae is carried out in a state with the opening closed, on the upper end part of the culturing tank 12, separately from the opening, a non-illustrated communication port is provided that places the interior and the exterior of the culturing tank 12 in communication with each other. Consequently, even in a state in which the opening is closed, the exhaust gas becomes capable of entering into and flowing out from the culturing tank 12 through the communication port.

Furthermore, the opening need not necessarily be provided at the upper end of the culturing tank 12. More specifically, the joined edge portions 26 may be provided on the entirety of the outer peripheral edge portion including the upper end of the culturing tank 12. In this case as well, a non-illustrated communication port that places the interior and the exterior of the culturing tank 12 in communication is provided on the upper end part of the culturing tank 12. In accordance with this feature, the exhaust gas becomes capable of entering into and flowing out from the culturing tank 12 through the communication port. In this manner, in the case that the joined edge portions 26 are provided on the entire outer peripheral edge of the culturing tank 12, for example, although neither of them is shown, a culturing solution supply port and a microalgae recovery port may be provided to the culturing tank 12. It becomes possible to supply the culturing solution L2 and the microalgae to the culturing tank 12 through the culturing solution supply port. Microalgae that have been cultured in the culturing tank 12 become capable of being recovered through the microalgae recovery port.

The culturing tank 12 is provided with partitioning members 32, joined portions 34, guide members 36, circulation portions 38, and the gas supply ports 30. According to the present embodiment, the culturing tank 12 includes two of the partitioning members 32, six of the joined portions 34, three of the guide members 36, six of the circulation portions 38, and three of the gas supply ports 30. However, the numbers of each of the partitioning members 32, the joined portions 34, the guide members 36, the circulation portions 38, and the gas supply ports 30 provided to the culturing tank 12 are not particularly limited.

Each of the partitioning members 32, the joined portions 34, the guide members 36, and the circulation portions 38 extend along the vertical direction (the upper/lower direction) in the interior of the culturing tank 12. It should be noted that the direction in which the partitioning members 32, the joined portions 34, the guide members 36, and the circulation portions 38 extend is not limited to being in parallel along the vertical direction, and may be along a direction inclined with respect to the vertical direction.

In the present embodiment, the interior of the culturing tank 12 is divided by the two partitioning members 32 into three individual regions 40 arranged alongside one another in the first horizontal direction (the direction of the arrows Y1 and Y2). By the regions 40 being arranged alongside one another in the first horizontal direction, the length of the culturing tank 12 in the first horizontal direction is longer than the length of the culturing tank 12 in the second horizontal direction (the direction of the arrows Z1 and Z2).

The partitioning members 32 are formed, for example, by joining inner wall surfaces of the culturing tank 12 to each other by fusion bonding. The respective regions 40 inside the culturing tank 12, which have been partitioned by the partitioning members 32, are further partitioned by the joined portions 34 formed by joining the inner wall surfaces of the culturing tank 12 themselves, for example, by fusion bonding. In accordance with this feature, each of the regions 40 includes one of the guide members 36 and two of the circulation portions 38 which are arranged alongside one another on both sides of the guide members 36 in the horizontal direction. Moreover, for example, in order to suppress concentration of stress, it is preferable for both of the end parts in the direction in which the partitioning members 32 and the joined portions 34 extend to be formed in an arcuate shape.

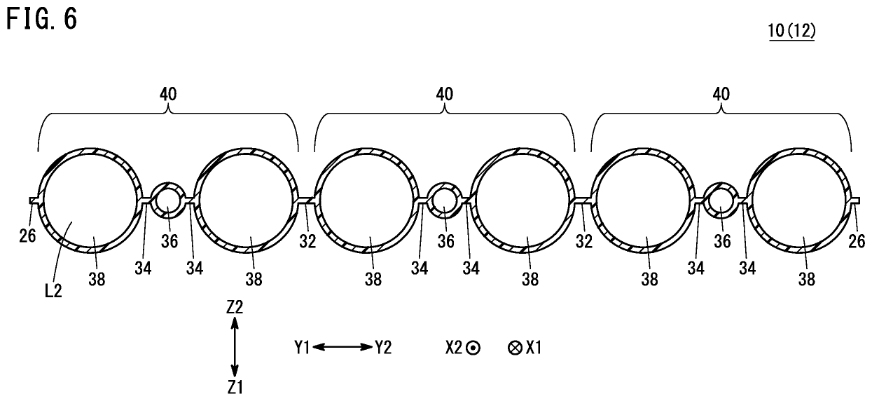

As shown in FIG. 6, in the case that the culturing solution L2 is accommodated in the culturing tank 12, the cross-sectional shape of each of the guide members 36 and the circulation portions 38 as viewed in the vertical direction is a substantially cylindrical shape. According to the present embodiment, the inner diameter of each of the circulation portions 38 as viewed in the vertical direction is greater than or equal to two times the inner diameter of the guide members 36 as viewed in the vertical direction, although the present invention is not particularly limited to this feature.

As shown in FIG. 5, the length of the joined portions 34 and the partitioning members 32 in the vertical direction (the direction of extension) is shorter than the length in the vertical direction of the culturing tank 12. Further, the length in the vertical direction of the partitioning members 32 is greater than or equal to the length in the vertical direction of the joined portions 34. At a more lower portion than the joined portions 34 inside the culturing tank 12, guide member inlets 42 are formed that allow communication between the guide members 36 and the circulation portions 38. Further, at a more upper part than the joined portions 34 inside the culturing tank 12, guide member outlets 44 are formed that allow communication between the guide members 36 and the circulation portions 38.

The gas supply ports 30 are disposed at the bottom of the culturing tank 12. The gas supply ports 30 are arranged below the guide members 36 that are provided in the respective regions 40 inside the culturing tank 12. As shown in FIG. 1, the gas supply ports 30 are connected to a gas supplying mechanism 50 via a gas supply pipe 48 provided with a supply fan 46. Therefore, by driving the supply fan 46, the supplied gas supplied from the gas supplying mechanism 50 is supplied to the interior of the culturing tank 12 through the gas supply pipe 48 and the gas supply ports 30.

As noted previously, the gas supply ports 30 are disposed downwardly of the guide members 36. Therefore, as shown in FIG. 5, the supplied gas which is supplied into the culturing tank 12 flows upwardly through the guide members 36. Consequently, in the respective regions 40 inside the culturing tank 12, a culturing solution flow F is generated in which the culturing solution L2 inside the circulation portions 38 flows into the guide members 36 from the guide member inlets 42, while in addition, the culturing solution L2 inside the guide members 36 flows out from the guide member outlets 44 into the circulation portions 38.

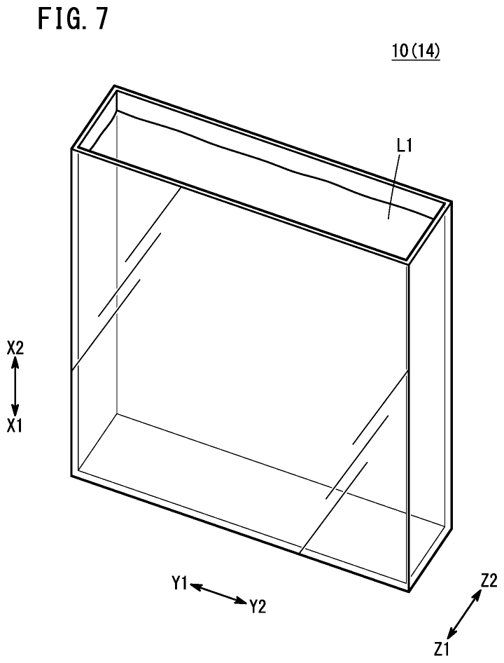

As shown in FIG. 7, similar to the culturing tank 12, the liquid storage tank 14 is formed, for example, from a flexible and light-transmitting material such as linear low density polyethylene (LLDPE) or the like. Moreover, the liquid storage tank 14 may be formed from a light-transmitting material such as acrylic resin, polycarbonate resin, glass, or the like. According to the present embodiment, the liquid storage tank 14 as a whole is formed from such a light-transmitting material. However, concerning the liquid storage tank 14, it is sufficient that at least the side surfaces (surfaces excluding the bottom surface and the upper surface) thereof are made of a light-transmitting material.

The liquid storage tank 14 has stored therein a stored liquid L1 which is supplied from a non-illustrated storage liquid supplying mechanism. The stored liquid L1 is a light-transmitting liquid such as water. As shown in FIGS. 1 to 4, the internal dimensions of the liquid storage tank 14 are set to be larger than the external dimensions of the culturing tank 12. Therefore, the culturing tank 12 can be installed in the interior of the liquid storage tank 14. In the interior of the liquid storage tank 14, for example, the opening (the communication port 28) at the upper end of the culturing tank 12 is fixed more on an upper side than the liquid surface of the stored liquid L1 inside the liquid storage tank 14. Consequently, a situation is avoided in which the stored liquid L1 is mixed into the culturing liquid L2 inside the culturing tank 12. Further, a situation is avoided in which the culturing solution L2 is mixed into the stored liquid L1 in the liquid storage tank 14.

Moreover, in FIGS. 1 to 4 and FIG. 7, etc., a casing-shaped liquid storage tank 14 is shown in which the upper end thereof is opened. However, together with storing the stored liquid L1 in the interior thereof, various shapes for the liquid storage tank 14 can be adopted that are capable of accommodating the culturing tank 12. The liquid storage tank 14 may be formed, for example, in the shape of a bag. Further, the culturing device 10 need not necessarily be equipped with the liquid storage tank 14.

As shown in FIGS. 1 to 4, the heat insulating portion 16 is formed from a material that possesses the light-transmitting property. The heat insulating portion 16 covers the side walls of the culturing tank 12. Consequently, the heat insulating portion 16 forms an air layer 52 that insulates the interior of the culturing tank 12. According to the present embodiment, the heat insulating portion 16 is formed in a sheet shape, for example, from a light-transmitting and flexible resin. As will be described later, the heat insulating portion 16 is supported by the support mechanism 18 (see FIGS. 2 to 4). The heat insulating portion 16 covers the side walls of the culturing tank 12 from an outer part of the side walls of the liquid storage tank 14. The heat insulating portion 16 and the side walls of the liquid storage tank 14 are arranged with a predetermined gap therebetween. Therefore, the air layer 52 is formed between the heat insulating portion 16 and the side walls of the liquid storage tank 14.

As shown in FIGS. 2 to 4, the heat insulating portion 16 includes a first heat insulating sheet 56 and a pair of second heat insulating sheets 58. As shown in FIG. 3 and FIG. 4, the first heat insulating sheet 56 includes a first portion 60, a second portion 62, and a third portion 64. The first portion 60 and the second portion 62 face toward each other while sandwiching therebetween the culturing tank 12, which is installed at the installation location, in the second horizontal direction (the direction of the arrows Z1 and Z2). Stated otherwise, the first portion 60 covers the side wall of the culturing tank 12 on the side of the arrow Z1. The second portion 62 covers the side wall of the culturing tank 12 on the side of the arrow Z2. The third portion 64 covers the upper surface (the communication port 28) of the culturing tank 12. An upper end of the first portion 60 and an upper end of the second portion 62 are continuous via the third portion 64. Therefore, the first heat insulating sheet 56 is a strip-shaped sheet with the first horizontal direction (the direction of the arrows Y1 and Y2) being the width direction. Moreover, a portion within the first heat insulating sheet 56 that extends more in the direction of the arrow Z1 than the first portion 60 may be fixed to the ground, for example, by a sheet fixing member 66. A portion of the first heat insulating sheet 56 that extends more in the direction of the arrow Z2 than the second portion 62 may be wound, for example, around a take-up roller 68.

As shown in FIG. 2, one of the pair of second heat insulating sheets 58 covers, in the direction of the arrow Y1, the side wall of the culturing tank 12 which is installed at the installation location. The other of the pair of second heat insulating sheets 58 covers the side wall of the culturing tank 12 in the arrow Y2 direction. Each of the second heat insulating sheets 58 is disposed in a manner so as to be capable of being stretched in the second horizontal direction. The material itself forming the second heat insulating sheets 58 may possess elasticity in the second horizontal direction. Further, a stretchable structure such as a bellows structure or a gathering structure (neither of which is shown) may be provided on the second heat insulating sheets 58 so as to be capable of being stretched and contracted in the second horizontal direction.

The support mechanism 18 supports the heat insulating portion 16 in a manner so that the first portion 60 and the second portion 62 can be moved relatively to approach toward or separate away from each other. More specifically, the support mechanism 18 includes two first support columns 70 and two second support columns 72. Each of the first support columns 70 and the second support columns 72 extend from the lower end of the culturing tank 12, which is installed at the installation location, along the vertical direction to an upper part which is higher than the upper end of the culturing tank 12. Moreover, it should be noted that the number of each of the first support columns 70 and the second support columns 72 is not particularly limited, and may be one support column or three or more support columns.

As shown in FIG. 2, the two first support columns 70 are arranged alongside one another with a gap along the width direction (the direction of the arrows Y1 and Y2) of the first portion 60. One of the two first support columns 70 is arranged at one end part (an end part in the direction of the arrow Y1) in the width direction of the first portion 60. The other of the two first support columns 70 is arranged at another end part (an end part in the direction of the arrow Y2) in the width direction of the first portion 60.

The two second support columns 72 (see FIGS. 3 and 4) are arranged alongside one another with a gap therebetween along the width direction of the second portion 62 (the direction of the arrows Y1 and Y2). One of the two second support columns 72 is arranged at one end part (an end part in the direction of the arrow Y1) in the width direction of the second portion 62. The other of the two second support columns 72 is arranged at another end part (an end part in the direction of the arrow Y2) in the width direction of the second portion 62. Moreover, it should be noted that one from among the two second support columns 72 which is arranged at the end part in the direction of the arrow Y1 does not appear in the drawings.

As shown in FIG. 3 and FIG. 4, at an end part in the direction of the arrow Y2, the first support columns 70 and the second support columns 72 face toward each other with a gap therebetween in the direction of the arrows Z1 and Z2. Between the upper ends of the first support columns 70 and the upper ends of the second support columns 72, expanding and retracting supporting parts 74 are provided that extend in the direction of the arrows Z1 and Z2. One end part (the end part in the direction of the arrow Z1) in the direction of extension of the expanding and retracting supporting parts 74 is fixed to the first support columns 70. The other end part (the end part in the direction of the arrow Z2) in the direction of extension of the expanding and retracting supporting parts 74 is fixed to the second support columns 72. The expanding and retracting supporting parts 74 have, for example, a nested structure in which an inner side shaft portion 78 is inserted into the inner side of an outer side tubular portion 76. Consequently, the expanding and retracting supporting parts 74 are constituted so as to be expandable and contractible in the direction of the arrows Z1 and Z2.

Moreover, although detailed illustration of this feature is omitted, the first support columns 70 and the second support columns 72 at the end part in the direction of the arrow Y1 are similar to the first support columns 70 and the second support columns 72 at the end part in the direction of the arrow Y2, in that they face toward each other with an interval therebetween in the direction of the arrows Z1 and Z2. Further, between the upper end part of the first support columns 70 and the upper end part of the second support columns 72 at the end part in the direction of the arrow Y1 as well, similar to between the upper end part of the first support columns 70 and the upper end part of the second support columns 72 at the end part in the direction of the arrow Y1, the expanding and retracting supporting parts 74 are provided so as to be capable of expanding and retracting in the direction of the arrows Z1 and Z2.

The first support columns 70 and the second support columns 72 are driven by the drive unit 22. Consequently, the first support columns 70 and the second support columns 72 are capable of approaching toward or separating away from each other along the second horizontal direction (the direction of the arrows Z1 and Z2). At this time, the culturing tank 12 and the liquid storage tank 14 are interposed between the first support columns 70 and the second support columns 72. When the drive unit 22 causes the first support columns 70 and the second support columns 72 to approach toward or separate away from each other, the expanding and retracting supporting parts 74 provided therebetween also expand and contract together.

Moreover, according to the present embodiment, both the first support columns 70 and the second support columns 72 are capable of being moved by the drive unit 22 in the second horizontal direction. However, the configuration for changing the distance in the horizontal direction between the first support columns 70 and the second support columns 72 is not limited to the aforementioned configuration. For example, either one of the first support columns 70 and the second support columns 72 may be fixed to the ground or the like. In this case, only the other one of the first support columns 70 and the second support columns 72 are capable of being moved by the drive unit 22 in the second horizontal direction. As the drive unit 22, a known configuration can be adopted which is capable of causing the first support columns 70 and the second support columns 72 to move in the manner described above, and therefore, a detailed description of this feature will be omitted.

As shown in FIGS. 2 to 4, a first lower end supporting part 82 is provided at the lower end part of the first support columns 70 with a fixing member 80 being interposed therebetween. A first upper end supporting part 84 is provided at the upper end part of the first support columns 70. At least one of the first lower end supporting part 82 and the first upper end supporting part 84 connects the parallel first support columns 70 by extending along the direction of the arrows Y1 and Y2. Moreover, it should be noted that in FIGS. 2 to 4, both of the first lower end supporting part 82 and the first upper end supporting part 84 connect the parallel first support columns 70 themselves by extending along the direction of the arrows Y1 and Y2.

Further, each of the first lower end supporting part 82 and the first upper end supporting part 84 is capable of being rotated with respect to the first support columns 70 with the direction of the arrows Y1 and Y2 serving as an axial direction. The first lower end supporting part 82 is fixed via the fixing member 80 to the first support columns 70. In accordance with this feature, the first lower end supporting part 82 is arranged to be separated further away from the liquid storage tank 14 (closer to the end part in the direction of the arrow Z1) than the first upper end supporting part 84.

A second lower end supporting part 86 is provided at the lower end part of the second support columns 72 with the fixing member 80 being interposed therebetween. A second upper end supporting part 88 is provided at the upper end part of the second support columns 72. At least one of the second lower end supporting part 86 and the second upper end supporting part 88 connects the parallel second support columns 72 by extending along the direction of the arrows Y1 and Y2. Moreover, it should be noted that in FIGS. 2 to 4, both of the second lower end supporting part 86 and the second upper end supporting part 88 connect the parallel second support columns 72 themselves by extending along the direction of the arrows Y1 and Y2.

Further, each of the second lower end supporting part 86 and the second upper end supporting part 88 is capable of being rotated with respect to the second support columns 72 with the direction of the arrows Y1 and Y2 serving as an axial direction. The second lower end supporting part 86 is fixed via the fixing member 80 to the second support columns 72. In accordance with this feature, the second lower end supporting part 86 is arranged to be separated further away from the liquid storage tank 14 (closer to the end part in the direction of the arrow Z2) than the second upper end supporting part 88.

The first heat insulating sheet 56 is supported by the support mechanism 18. Consequently, the first portion 60 extends from the first lower end supporting part 82 toward the first upper end supporting part 84. Further, the second portion 62 extends from the second upper end supporting part 88 toward the second lower end supporting part 86. Furthermore, the third portion 64 extends in the second horizontal direction from the first upper end supporting part 84 toward the second upper end supporting part 88.

According to the present embodiment, the first heat insulating sheet 56 extends from the sheet fixing member 66 along the second horizontal direction, and thereafter, by coming into contact with a lower part of the first lower end supporting part 82, the direction of extension thereof is changed to the vertical direction. Further, by coming into contact with an upper part of the first upper end supporting part 84, the first heat insulating sheet 56 changes the direction of extension to the second horizontal direction. By coming into contact with an upper part of the second upper end supporting part 88, the first heat insulating sheet 56 changes the direction of extension to the vertical direction. Furthermore, by coming into contact with a lower part of the second lower end supporting part 86, the first heat insulating sheet 56 changes the direction of extension to the second horizontal direction.

In the foregoing manner, in a state in which the first heat insulating sheet 56 is supported by the support mechanism 18, as shown in FIG. 3, the drive unit 22 causes the first support columns 70 and the second support columns 72 to approach toward each other. In accordance with this feature, the first portion 60 and the second portion 62 are capable of approaching toward each other. As a result, the air layer 52 formed between the first heat insulating sheet 56 and the side walls of the culturing tank 12 can be made thinner.

On the other hand, as shown in FIG. 4, the drive unit 22 causes the first support columns 70 and the second support columns 72 to separate away from each other, whereby the first portion 60 and the second portion 62 can be made to separate away from each other. As a result, the air layer 52 formed between the first heat insulating sheet 56 and the side walls of the culturing tank 12 can be made thicker.

Moreover, each of the first lower end supporting part 82, the first upper end supporting part 84, the second lower end supporting part 86, and the second upper end supporting part 88 are capable of rotating while being in contact with the first heat insulating sheet 56. Consequently, a frictional force generated between the first heat insulating sheet 56 and the support mechanism 18 can be reduced, and the first portion 60 and the second portion 62 can be smoothly made to approach toward or separate away from each other.

Further, as noted previously, by the first portion 60 and the second portion 62 being brought into close proximity, the length of the third portion 64 may become excessive with respect to the distance between the first support columns 70 and the second support columns 72. In this case, for example, the take-up roller 68 is rotated in a direction to take up the first heat insulating sheet 56. Consequently, any excessive portion can be eliminated, and it becomes possible to cause the first heat insulating sheet 56 to satisfactorily fit along the sides and the upper surface of the culturing tank 12.

On the other hand, as noted previously, by the first portion 60 and the second portion 62 being separated away from each other, the length of the third portion 64 may become insufficient with respect to the distance between the first support columns 70 and the second support columns 72. In this case, for example, the take-up roller 68 is rotated in a direction in which the first heat insulating sheet 56 is drawn out. Consequently, any insufficient portion can be eliminated, and it becomes possible to cause the first heat insulating sheet 56 to satisfactorily fit along the sides and the upper surface of the culturing tank 12.

Instead of causing the take-up roller 68 to be rotated, or alternatively, in conjunction with causing the take-up roller 68 to be rotated in the manner described above, the first heat insulating sheet 56 which is formed from an elastic material may be stretched or retracted in accordance with the distance between the first support columns 70 and the second support columns 72. In accordance with this feature as well, it is possible to eliminate any excess or deficiency in the lengths of the first portion 60, the second portion 62, and the third portion 64 that occurs accompanying the first portion 60 and the second portion 62 being brought into proximity or being separated away from each other.

One of the pair of second heat insulating sheets 58 is supported by the expanding and retracting supporting parts 74 at the end part in the direction of the arrow Y1, thereby covering the side wall of the culturing tank 12 (the liquid storage tank 14) at the end part in the direction of the arrow Y1. The other of the pair of second heat insulating sheets 58 is supported by the expanding and retracting supporting parts 74 at the end part in the direction of the arrow Y2, thereby covering the side wall of the culturing tank 12 (the liquid storage tank 14) at the end part in the direction of the arrow Y2. Further, each of the second heat insulating sheets 58 expands and contracts in the second horizontal direction, in accordance with the expanding and contracting of the expanding and retracting supporting parts 74. In accordance therewith, even if the first support columns 70 and the second support columns 72 are moved to approach toward or separate away from each other in the manner described above, a state is maintained in which both of the side walls of the culturing tank 12 in the first horizontal direction are covered by the second heat insulating sheets 58.

The heat insulating portion 16 is supported by the support mechanism 18 in the manner described above, thereby forming a closed space 90 in which the culturing tank 12 is accommodated. As shown in FIG. 1, a pipe insertion opening 92 is provided in a lower part of the heat insulating portion 16. The gas supply pipe 48 for connecting the gas supply ports 30 of the culturing tank 12 and the gas supplying mechanism 50, which is provided on the outer side of the closed space 90, is inserted through the pipe insertion opening 92.

A gas discharge port 94 is provided in an upper part of the heat insulating portion 16. The gas discharge port 94 makes it possible for the exhaust gas discharged from the culturing tank 12 into the closed space 90 via the communication port 28 to be discharged from the closed space 90. One end part of a gas recovery pipe 98 is connected to the gas discharge port 94. An exhaust fan 96 is provided to the gas recovery pipe 98. By the exhaust fan 96 being driven, the gas recovery pipe 98 recovers the exhaust gas discharged from the gas discharge port 94. The other end of the gas recovery pipe 98 is connected to the gas supply pipe 48 at a more upstream location than the supply fan 46. Therefore, the exhaust gas recovered by the gas recovery pipe 98 is capable of being supplied to the culturing solution L2 inside the culturing tank 12 via the gas supply pipe 48 and the gas supply ports 30.

As shown in FIGS. 3 and 4, the temperature sensor 20 is disposed in the interior of the closed space 90, for example. The temperature sensor 20 measures the temperature of the culturing solution L2 inside the culturing tank 12. Moreover, it should be noted that the temperature sensor 20 may be of a contact type that measures the temperature by coming into contact with the culturing solution L2 inside the culturing tank 12. The temperature sensor 20 may also be of a non-contact type that measures the temperature without coming into contact with the culturing solution L2. The measured value of the temperature sensor 20 is delivered to the control unit 24.

The control unit 24 is configured, for example, in the form of a microcomputer including a non-illustrated CPU or the like. The control unit 24 performs various processes and controls in relation to the culturing device 10, by executing predetermined calculations in accordance with a control program. The control unit 24 controls the drive unit 22 in a manner so that the distance (the thickness of the air layer 52) between the first portion 60 and the second portion 62 becomes of a length associated with the measured value of the temperature sensor 20.

For example, the higher the measured value of the temperature sensor 20 is, the control unit 24 causes the first portion 60 and the second portion 62 to be placed in closer proximity to each other. In accordance with this feature, the air layer 52 formed between the first heat insulating sheet 56 and the side walls of the culturing tank 12 is made thinner. Consequently, the heat insulation of the air layer 52 is reduced. As a result, it is easier for the heat of the culturing solution L2 inside the culturing tank 12 to escape to the outer side of the closed space 90. On the other hand, the smaller the measured value of the temperature sensor 20 is, the more the first portion 60 and the second portion 62 are separated away from each other. In accordance with this feature, the air layer 52 formed between the first heat insulating sheet 56 and the side walls of the culturing tank 12 is made thicker. Consequently, the heat insulation of the air layer 52 is increased. As a result, the heat of the culturing solution L2 inside the culturing tank 12 is prevented from being transferred to the outer side of the closed space 90. Further, apart from the measured value of the temperature sensor 20, the control unit 24, for example, may adjust the thickness of the air layer 52 based on the sunlight intensity detected by a non-illustrated sunlight sensor. The control unit 24 may adjust the thickness of the air layer 52 on the basis of a calendar or the like that has been set beforehand.

The culturing device 10 according to the present embodiment is basically configured in the manner described above. A description will be given concerning an example of a method of culturing microalgae using the culturing device 10. In the case of culturing microalgae using the culturing device 10, first, as shown in FIGS. 1 to 4, the culturing tank 12 is arranged inside the stored liquid L1 of the liquid storage tank 14. In this state, the culturing solution L2, which is supplied from a non-illustrated culturing solution supply mechanism, is accommodated in the interior of the culturing tank 12. The culturing liquid L2 is supplied into the culturing tank 12 inside the stored liquid L1. In accordance with this feature, it is possible to prevent the culturing tank 12 from being damaged by the hydraulic pressure of the culturing solution L2.

Next, as shown in FIG. 1 and FIG. 5, by driving the supply fan 46, the supplied gas, which is supplied from the gas supplying mechanism 50, is supplied via the gas supply pipe 48 and the gas supply ports 30 toward the guide members 36 of each of the regions 40 inside the culturing tank 12. Consequently, as shown in FIG. 5, the culturing solution flow F can be generated in each of the regions 40 of the culturing tank 12. Therefore, the microalgae can be caused to circulate in the culturing tank 12 together with the culturing solution L2. In accordance with this feature, the microalgae can be satisfactorily dispersed. Consequently, the supplied gas or light or the like can be effectively supplied to the entirety of the microalgae.

The stored liquid L1 has the light-transmitting property. The heat insulating portion 16 is constituted from a light-transmitting material. The side walls of each of the culturing tank 12 and the liquid storage tank 14 are constituted from a light-transmitting material. Therefore, it is possible to irradiate the microalgae with light such as sunlight through the heat insulating portion 16 and the side walls of the culturing tank 12 and the liquid storage tank 14. Consequently, it is possible to ensure a large light receiving area with respect to the volume in which the microalgae are cultured, as compared with culturing that takes place in a so-called open pond (raceway pond). As a result, it becomes possible to distribute the light energy in which an excess or deficiency of light is suppressed with respect to a greater amount of the microalgae inside the culturing tank 12.

The microalgae perform photosynthesis using the carbon dioxide within the supplied gas, light, and the water within the culturing solution L2. In accordance with this feature, the cells grow and propagate while starch and the like is accumulated inside the cells. A surplus amount of the supplied gas which is not used for photosynthesis is discharged from the culturing tank 12 into the closed space 90 via the communication port 28, and becomes the exhaust gas. Stated otherwise, the exhaust gas contains carbon dioxide gas. Therefore, by forming the closed space 90 by surrounding the culturing tank 12 with the heat insulating portion 16, the concentration of the carbon dioxide gas around the periphery of the culturing tank 12 can be increased. Consequently, it becomes easier for the carbon dioxide gas inside the closed space 90 to be supplied to the culturing solution L2 again through the communication port 28. Therefore, it becomes possible to enhance the efficiency with which the carbon dioxide gas supplied from the gas supplying mechanism 50 is utilized.

When the exhaust gas discharged from the communication port 28 exceeds the volume of the closed space 90, the exhaust gas is discharged from the closed space 90. In this case, by the exhaust fan 96 being driven, the exhaust gas that is discharged into the closed space 90 via the communication port 28 is recovered into the gas recovery pipe 98 through the gas discharge port 94 provided in an upper part of the heat insulating portion 16. Under a driving action of the supply fan 46, the exhaust gas that was recovered into the gas recovery pipe 98 is supplied again to the culturing solution L2 inside the culturing tank 12 via the gas supply pipe 48 and the gas supply ports 30. Owing to this feature as well, it also becomes possible to enhance the efficiency with which the carbon dioxide gas is utilized.

Further, in the case of culturing the microalgae in the culturing tank 12 in the manner described above, the temperature sensor 20 measures the temperature of the culturing solution L2 inside the culturing tank 12. Further, the drive unit 22 is controlled by the control unit 24 based on the measured value of the temperature sensor 20. Consequently, the air layer 52 formed between the side walls of the culturing tank 12 and the first heat insulating sheet 56 is adjusted to have a thickness suitable for culturing the microalgae. For example, the thickness of the air layer 52 may be adjusted based on the sunlight intensity and the calendar, in a manner so that the inside of the culturing tank 12 is maintained in an environment suitable for culturing the microalgae.

It should be noted that the drive unit 22 is not limited to being controlled by the control unit 24. The drive unit 22 may be configured in a manner so that the thickness of the air layer 52 can be adjusted by an operation of an operator. Further, the support mechanism 18 is not limited to being driven by the drive unit 22. The support mechanism 18 may be configured in a manner to enable the thickness of the air layer 52 to be adjusted by the operator manually adjusting the distance between the first support columns 70 and the second support columns 72.

As noted previously, the culturing tank 12 is arranged inside the stored liquid L1 that is stored in the liquid storage tank 14. Therefore, the culturing solution L2 and the microalgae inside the culturing tank 12 are prevented from receiving an influence of changes in the external environment (for example, the external temperature, the intensity of solar radiation, the amount of solar radiation, or the duration of solar radiation) of the culturing device 10. Due to such features, it becomes easy to maintain the temperature of the culturing solution L2 inside the culturing tank 12 at a temperature that is suitable for culturing the microalgae.

By carrying out culturing of the microalgae in the manner described above, the microalgae are sufficiently propagated within the culturing tank 12. Thereafter, for example, the microalgae are collected from the interior of the culturing tank 12 together with the culturing solution L2 via the communication port 28 that is exposed from the heat insulating portion 16. Thereafter, the microalgae can be obtained by separating the microalgae from the culturing solution L2.

From the foregoing, in the culturing device 10 according to the present embodiment, the side walls of the light-transmitting culturing tank 12 are covered by the light-transmitting heat insulating portion 16. The interior of the culturing tank 12 is insulated by the air layer 52 which is formed by the heat insulating portion 16. Each of the heat insulating portion 16, the air layer 52, and the side walls possess the light-transmitting property. Therefore, the light can be satisfactorily shone into the culturing tank 12 through the heat insulating portion 16, the air layer 52, and the side walls. As a result, it is possible to increase the heat insulation property inside the culturing tank 12, while at the same time increasing the ratio of the light receiving area with respect to the volume in which the microalgae are cultured. Consequently, it becomes possible for the microalgae to be satisfactorily cultured.

With the culturing device 10 according to the above-described embodiment, the heat insulating portion 16 is in the form of a flexible sheet, and the air layer 52 is formed between the side walls and the heat insulating portion 16 which are arranged with an interval therebetween. In accordance with such features, by a simple structure in which the flexible and sheet shaped heat insulating portion 16 is arranged with an interval from the side walls of the culturing tank 12, the heat insulation in the culturing tank 12 can be enhanced without hindering the shining of light onto the microalgae.

In the culturing device 10 according to the above-described embodiment, the heat insulating portion 16 covers the side walls of the culturing tank 12 and the upper surface of the culturing tank 12. In accordance with this feature, the air layer 52 is also formed on the upper surface of the culturing tank 12 by the heat insulating portion 16, and the heat insulation property inside the culturing tank 12 can be increased.

The culturing tank 12 of the culturing device 10 according to the above-described embodiment is provided with the gas supply ports 30, which are capable of supplying the supplied gas to the culturing solution L2 inside the culturing tank 12, the upper surface of the culturing tank 12 is provided with the communication port 28, which places the interior and the exterior of the culturing tank 12 in communication, the heat insulating portion 16 forms the closed space 90 in which the culturing tank 12 is accommodated on the inner side thereof, and an upper part of the heat insulating portion 16 is provided with the gas discharge port 94, which enables the exhaust gas discharged from the culturing tank 12 into the closed space 90 through the communication port 28 to be discharged from the closed space 90.

By the closed space 90 being formed by the heat insulating portion 16 in this manner, the thermal insulation inside the culturing tank 12 can be increased, and together therewith, the carbon dioxide gas contained in the exhaust gas can easily be retained around the periphery of the culturing tank 12. Therefore, it becomes possible to supply again the carbon dioxide gas contained in the exhaust gas to the culturing solution L2 inside the culturing tank 12 via the communication port 28. Therefore, the efficiency with which the carbon dioxide gas supplied from the gas supplying mechanism 50 is utilized can be enhanced.

Further, the gas discharge port 94 is provided in an upper part of the heat insulating portion 16. Therefore, it is possible to prevent the heat insulating portion 16 from being damaged by the pressure of the exhaust gas that is discharged into the closed space 90. Carbon dioxide gas is heavier than air, and there is a tendency for the carbon dioxide gas to be collected downwardly of the closed space 90. Therefore, by the gas discharge port 94 being provided on an upper part of the heat insulating portion 16, it is possible to relatively easily increase the concentration of the carbon dioxide gas inside the closed space 90.

In the culturing device 10 according to the above-described embodiment, the gas supply ports 30 are connected to the gas supplying mechanism 50 via a gas supply pipe 48, one end part of the gas recovery pipe 98, which recovers the exhaust gas discharged from the gas discharge port 94, is connected to the gas discharge port 94, and another end part of the gas recovery pipe 98 is connected to the gas supply pipe 48, and the exhaust gas recovered into the gas recovery pipe 98 is supplied to the culturing solution L2 inside the culturing tank 12 through the gas supply pipe 48 and the gas supply ports 30.

In accordance with such features, the carbon dioxide gas contained in the exhaust gas which is discharged from the closed space 90 can be supplied to the gas supply ports 30 of the culturing tank 12. More specifically, it becomes possible for the carbon dioxide to be circulated between the culturing tank 12 and the closed space 90. Therefore, it is possible to further effectively enhance the efficiency with which the carbon dioxide gas supplied from the gas supplying mechanism 50 is utilized.

In the culturing device 10 according to the above-described embodiment, there is further provided the support mechanism 18 that supports the heat insulating portion 16, the heat insulating portion 16 includes the first portion 60 and the second portion 62 that face toward one another while sandwiching therebetween in the horizontal direction the culturing tank 12 which is installed at the installation location, and the support mechanism 18 supports the heat insulating portion 16 in a manner so that the first portion 60 and the second portion 62 are capable of approaching toward or separating away from each other.

In accordance with such features, the distance between the first portion 60 and the second portion 62 can be adjusted by the support mechanism 18. Consequently, the thickness of the air layer 52 that is formed between the heat insulating portion 16 and the side walls of the culturing tank 12 can be adjusted. Therefore, for example, the thickness of the air layer 52 can be adjusted in accordance with the temperature of the culturing solution L2 inside the culturing tank 12, and to match with the environment (the external environment) of the installation location where the culturing tank 12 is installed. Consequently, it becomes easy to maintain the environment inside the culturing tank 12 in an environment that is suitable for culturing the microalgae.

In the culturing device 10 according to the above-described embodiment, the support mechanism 18 includes the first support columns 70 and the second support columns 72, each of the first support columns 70 and the second support columns 72 extends along the vertical direction from the lower end part of the culturing tank 12 which is installed at the installation location, to an upper part which is higher than the upper end of the culturing tank 12, the lower end part of the first support columns 70 is provided with the first lower end supporting part 82, and the upper end part of the first support columns 70 is provided with the first upper end supporting part 84, the lower end part of the second support columns 72 is provided with the second lower end supporting part 86, and the upper end part of the second support columns 72 is provided with the second upper end supporting part 88, the first support columns 70 and the second support columns 72 are capable of relatively approaching toward or separating away from each other along the horizontal direction in a state with the culturing tank 12 being interposed therebetween, and the first portion 60 extends from the first lower end supporting part 82 to the first upper end supporting part 84, the second portion 62 extends from the second upper end supporting part 88 to the second lower end supporting part 86, and the first portion 60 and the second portion 62 are continuous via the third portion 64 that extends in the horizontal direction from the first upper end supporting part 84 toward the second upper end supporting part 88.

In accordance with such features, the distance between the first portion 60 and the second portion 62 can be easily adjusted by causing the first support columns 70 and the second support columns 72 to approach toward or separate away from each other. Hence, the thickness of the air layer 52 that is formed between the heat insulating portion 16 and the side walls of the culturing tank 12 can be easily adjusted.

In the culturing device 10 according to the above-described embodiment, the first support column 70 comprises a plurality of first support columns 70 that are disposed in parallel with one another along the width direction of the first portion 60, at least one of the first lower end supporting part 82 and the first upper end supporting part 84 extends along the width direction of the first portion 60, and connects the first support columns 70 disposed in parallel, the second support columns 72 comprises a plurality of second support columns 72 and are disposed in parallel with one another along the width direction of the second portion 62, and at least one of the second lower end supporting part 86 and the second upper end supporting part 88 extends along the width direction of the second portion 62, and connects the second support columns 72 disposed in parallel.

In accordance with such features, the first heat insulating sheet 56 of the heat insulating portion 16 can be satisfactorily supported by at least one of the first lower end supporting part 82 and the first upper end supporting part 84 that extend along the width direction of the first portion 60. Similarly, the first heat insulating sheet 56 of the heat insulating portion 16 can be satisfactorily supported by at least one of the second lower end supporting part 86 and the second upper end supporting part 88 that extend along the width direction of the second portion 62.

Further, deformation or the like of the support mechanism 18 is suppressed by connecting the parallel first support columns 70 themselves to each other by at least one of the first lower end supporting part 82 and the first upper end supporting part 84. Further, deformation or the like of the support mechanism 18 is suppressed by connecting the parallel second support columns 72 themselves to each other by at least one of the second lower end supporting part 86 and the second upper end supporting part 88. More specifically, it becomes possible for the support mechanism 18 to be constituted in a firm manner. Therefore, it is possible to satisfactorily maintain the state in which the heat insulating portion 16 is supported by the support mechanism 18.

In the culturing device 10 according to the above described embodiment, there are further provided the temperature sensor 20 that measures the temperature of the culturing solution L2 inside the culturing tank 12, the drive unit 22 that drives the first portion 60 and the second portion 62 in directions to approach toward or separate away from each other, and the control unit 24 that controls the drive unit 22 in a manner so as to adjust the distance between the first portion 60 and the second portion 62 in accordance with the measured value of the temperature sensor 20. In accordance with such features, the distance between the first portion 60 and the second portion 62 can be automatically adjusted by the control unit 24 based on the measurement result of the temperature sensor 20. Therefore, it becomes easier to maintain the environment inside the culturing tank 12 in an environment that is suitable for culturing the microalgae.

It should be understood that the present invention is not limited to the embodiment described above, and various additional or modified configurations could be adopted therein without departing from the essence and gist of the present invention.

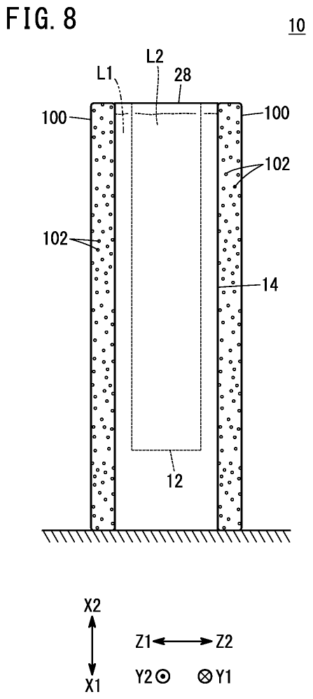

For example, in the above-described embodiment, the heat insulating portion 16 is in the form of a flexible sheet, and the air layer 52 is formed between the side walls and the heat insulating portion 16 which are arranged with an interval therebetween. However, the present invention is not particularly limited to this feature. For example, instead of the heat insulating portion 16 shown in FIGS. 1 to 4, the culturing device 10 may be equipped with a heat insulating portion 100 shown in FIG. 8. The heat insulating portion 100 shown in FIG. 8, for example, is an aerogel having a heat insulating property and permeability, such as a silica aerogel. The heat insulation portion 100 is arranged along the side walls of the culturing tank 12. In accordance with this feature, an air layer 102 that insulates the interior of the culturing tank 12 is formed within the pores of the aerogel.

In this manner, even in the case that the heat insulating portion 100 is made of an aerogel, light can be satisfactorily irradiated into the culturing tank 12 via the heat insulating portion 100, the air layer 102, and the side walls. Therefore, it is possible to increase the heat insulation property inside the culturing tank 12, while at the same time increasing the ratio of the light receiving area with respect to the volume in which the microalgae are cultured. Consequently, it is possible for the microalgae to be satisfactorily cultured. In addition, in this case, the heat insulating property inside the culturing tank 12 can be increased by a simple configuration in which the heat insulating portion 100 is arranged along the side walls of the culturing tank 12. Further, since the aerogel is relatively lightweight, even in the case that the heat insulating portion 100 falls over the culturing tank 12, and the heat insulating portion 100 collides with the culturing tank 12, damage to the culturing tank 12 can be avoided.

In the above-described embodiment, in the interior of the culturing tank 12, the guide members 36 and the circulation portions 38, which extend along the vertical direction in the case that the culturing tank 12 is installed at the installation location, are provided alongside one another in the horizontal direction. The guide members 36 and the circulation portions 38 communicate with each other through the guide member inlets 42 provided in the lower part in the vertical direction, and the guide member outlets 44 provided in the upper part in the vertical direction. The gas supply ports 30, which enable the gas to be supplied to the guide members 36 upwardly from below, are provided in the bottom of the culturing tank 12. When the gas is supplied to the guide members 36 from the gas supply ports 30, the culturing solution flow F is generated in which the culturing solution L2 inside the circulation portions 38 flows into the guide members 36 from the guide member inlets 42, and the culturing solution L2 inside the guide members 36 flows out from the guide member outlets 44 into the circulation portions 38.

In accordance with such features, with a simple configuration in which the gas necessary for culturing the microalgae is supplied from the gas supply ports 30 and made to flow through the guide members 36, the culturing solution flow F can be generated inside the culturing tank 12. In addition, for example, there is no need to provide and to drive a specialized configuration such as a water pump in order to cause the culturing solution flow F to be generated. Accordingly, with a simple configuration, it becomes possible for the microalgae to be satisfactorily cultured, while suppressing an increase in energy consumption.

However, the configuration of the culturing tank 12 is not particularly limited. For example, in the culturing tank 12, the culturing liquid L2 inside the culturing tank 12 may be circulated by generating a culturing solution flow by a non-illustrated water pump.

1. A culturing device having a culturing tank side walls of which are made from a light-transmitting material, and configured to culture microalgae in a culturing solution accommodated in the culturing tank and which is irradiated with light through the side walls, the culturing device comprising
a liquid storage tank that is formed from a light-transmitting material and stores a stored liquid having a light-transmitting property,
wherein
the culturing tank is arranged inside the stored liquid stored in the liquid storage tank, and includes a light-transmitting heat insulating portion configured to cover the liquid storage tank, and
the heat insulating portion forms an air layer that insulates an interior of the culturing tank.

2. The culturing device according to claim 1, wherein:
the heat insulating portion is an aerogel; and
the air layer is formed within pores of the aerogel.

3. The culturing device according to claim 1, wherein:
the heat insulating portion is in the form of a sheet that possesses flexibility; and
the air layer is formed between the liquid storage tank and the heat insulating portion that are arranged with a space therebetween.

4. The culturing device according to claim 3, wherein the heat insulation portion covers side walls of the liquid storage tank, an upper surface of the liquid storage tank, and an upper surface of the culturing tank.

5. The culturing device according to claim 4, wherein:
the culturing tank is provided with a gas supply port configured to be capable of supplying a supplied gas to the culturing solution inside the culturing tank;
the upper surface of the culturing tank is provided with a communication port configured to place the interior and the exterior of the culturing tank in communication,
the heat insulating portion forms a closed space in which the culturing tank is accommodated on an inner side thereof; and
an upper part of the heat insulating portion is provided with a gas discharge port configured to enable the exhaust gas discharged from the culturing tank into the closed space through the communication port to be discharged from the closed space.

6. The culturing device according to claim 5, wherein:
the gas supply port is connected to a gas supplying mechanism via a gas supply pipe;
one end part of a gas recovery pipe configured to recover the exhaust gas discharged from the gas discharge port is connected to the gas discharge port, and another end part of the gas recovery pipe is connected to the gas supply pipe; and
the exhaust gas recovered into the gas recovery pipe is supplied to the culturing solution inside the culturing tank through the gas supply pipe and the gas supply port.

7. The culturing device according to claim 3, further comprising:
a support mechanism configured to support the heat insulating portion;
the heat insulating portion includes a first portion and a second portion configured to face toward one another while sandwiching therebetween in a horizontal direction the culturing tank that is installed at an installation location; and
the support mechanism supports the heat insulation portion in a manner so that the first portion and the second portion are capable of approaching toward or separating away from each other.

8. The culturing device according to claim 7, wherein:
the support mechanism includes a first support column and a second support column;
each of the first support column and the second support column extends along a vertical direction from a lower end part of the culturing tank installed at the installation location to an upper part that is higher than an upper end of the culturing tank;
a lower end part of the first support column is provided with a first lower end supporting part, and an upper end part of the first support column is provided with a first upper end supporting part;
a lower end part of the second support column is provided with a second lower end supporting part, and an upper end part of the second support column is provided with a second upper end supporting part;
the first support column and the second support column are capable of relatively approaching toward or separating away from each other along a horizontal direction in a state with the culturing tank being interposed therebetween; and
the first portion extends from the first lower end supporting part to the first upper end supporting part, the second portion extends from the second upper end supporting part to the second lower end supporting part, and the first portion and the second portion are continuous via a third portion configured to extend in a horizontal direction from the first upper end supporting part toward the second upper end supporting part.

9. The culturing device according to claim 8, wherein:
the first support column comprises a plurality of first support columns that are disposed in parallel with one another along the width direction of the first portion;
at least one of the first lower end supporting part and the first upper end supporting part extends along the width direction of the first portion, and connects the first support columns disposed in parallel;
the second support column comprises a plurality of second support columns and are disposed in parallel with one another along the width direction of the second portion; and
at least one of the second lower end supporting part and the second upper end supporting part extends along the width direction of the second portion, and connects the second support columns disposed in parallel.

10. The culturing device according to claim 7, further comprising:
a temperature sensor configured to measure a temperature of the culturing solution inside the culturing tank;
a drive unit configured to drive the first portion and the second portion in directions to approach toward or separate away from each other; and
a control unit configured to control the drive unit in a manner so as to adjust the distance between the first portion and the second portion in accordance with a measured value of the temperature sensor.
